(19) United States
(12) Patent Application Publication
(10) Pub. No.: US 2009/0131761 A1
(43) Pub. Date: May 21, 2009

(54) DEVICE PROVIDING SPOT-CHECK OF VITAL SIGNS USING AN IN-THE-EAR PROBE

(75) Inventors: Richard M. Moroney III, Princeton, NJ (US); Larry Nielsen, Burlington, MA (US); Christopher J. Poux, Mercerville, NJ (US)

Correspondence Address: PHILIPS INTELLECTUAL PROPERTY & STANDARDS, P. O. Box 3001, BRIARCLIFF MANOR, NY, 10510, US

(73) Assignee: KONINKLIJKE PHILIPS ELECTRONICS N. V., Eindhoven (NL)

(21) Appl. No.: 11/995,008

(22) PCT Filed: Jun. 20, 2006
(86) PCT No.: PCT/IB2006/051994

§ 371 Date: Nov. 13, 2008

Related U.S. Application Data
(60) Provisional application No. 60/695,725, filed on Jun. 30, 2005.
(60) Provisional application No. 60/777,502, filed on Feb. 28, 2006.

(51) Int. Cl. A61B 5/145; A61B 5/02

(52) U.S. Cl. 600/301

(57) ABSTRACT

A portable physiological monitoring device (12) includes a receiver (22) that wirelessly receives physiological measurements from each of a plurality of in-the-ear probes (14) upon entering a communication range of one of the in-the-ear probes (14). The portable physiological monitoring device (12) farther includes a display (30) for presenting the physiological measurements.

The following relates to monitoring physiological parameters. It finds particular application as a portable device that receives physiological measurements such as blood pressure, respiration, perfusion index, blood oxygen, pulse rate, body temperature, etc. from an in-the-ear probe, displays the physiological measurement, and conveys the physiological measurement to a monitoring station.

Physiological parameters have been measured from within the ear via an in-the-ear probe. One such probe includes a multi-parameter physiological measurement system that non-invasively measures blood pressure as well as respiration, perfusion, blood oxygen, pulse rate, body temperature, etc. from within the ear canal. This probe includes a series of in-the-ear sensors that interconnect to electronics and a battery pack that are mounted behind the ear or in connection with another location on the patient (e.g., around the neck, wrist, etc.). A processor in the electronics analyzes the raw data and converts it into measurements of physiological parameters that are wirelessly sent to a central monitoring station, which is remote form the location of the subject being monitored.

Typically, such physiological parameters are continuously or periodically measured and conveyed to the central monitoring station. However, in some instances it is not convenient for a clinician to have to view the parameters at the central monitoring station, which is located away from the patient. In addition, instances exist wherein continuous and/or periodic conveyance of such information is not desirable. For example, spot-check or on-demand monitoring may be more desirable with patients having their vital signs checked only every one, two, four . . . hours. In another example, the network used for such conveyance may have limited bandwidth that is shared with other wireless monitoring devices. Such devices may have to compete for available bandwidth, which may result in delays and/or lost data. In yet another example, the sensitivity of the information may dictate how often it is transmitted, if at all.

In one aspect, a portable physiological monitoring device is illustrated. The portable physiological monitoring device includes a receiver and a display. The receiver wirelessly receives physiological measurements from each of a plurality of in-the-ear probes upon entering a communication range of one of the in-the-ear probes. The received physiological measurements are subsequently presented on the display.

One advantage resides in locally displaying physiological signals measured with an in-the-ear probe.

Another advantage is user validation of physiological signals measured with an in-the-ear probe.

Another advantage is that spot-check monitoring of the physiological signals measured with an in-the-ear probe is facilitated.

Another advantage is using the device as a continuous monitor for the physiological signals measured with an in-the-ear probe with or without the use of a central monitoring station.

Still further advantages will become apparent to those of ordinary skill in the art upon reading and understanding the detailed description of the preferred embodiments.

The drawings are only for purposes of illustrating embodiments and are not to be construed as limiting the claims.

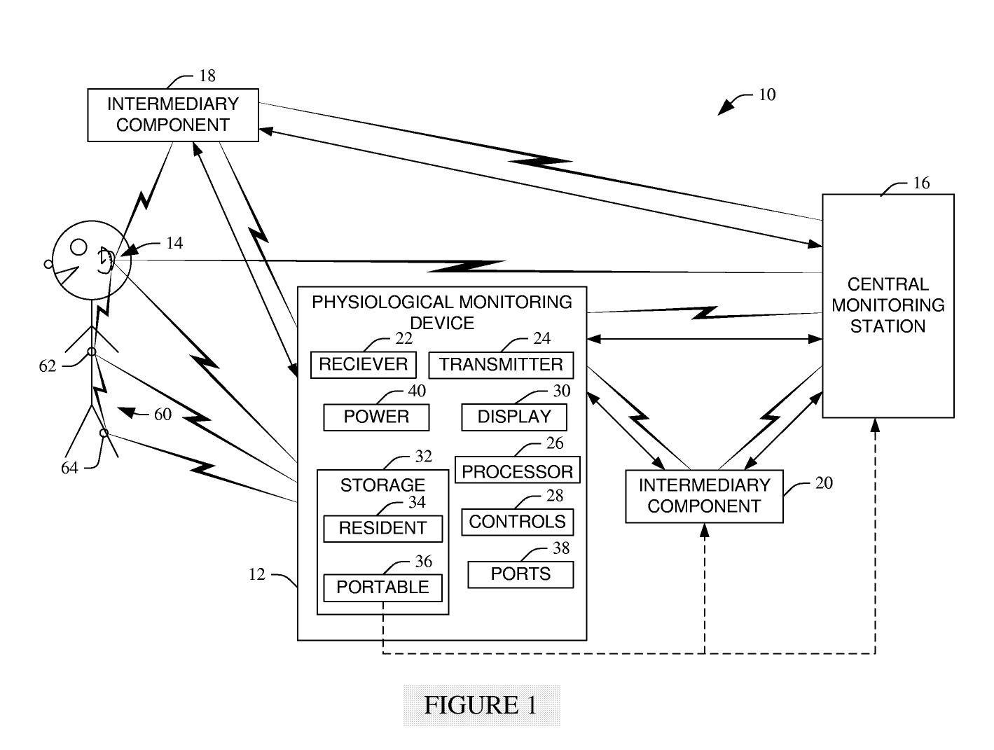

FIG. 1 illustrates an exemplary physiological monitoring device that communicates with an in-the-ear physiological measurement probe and other physiological monitoring equipment.

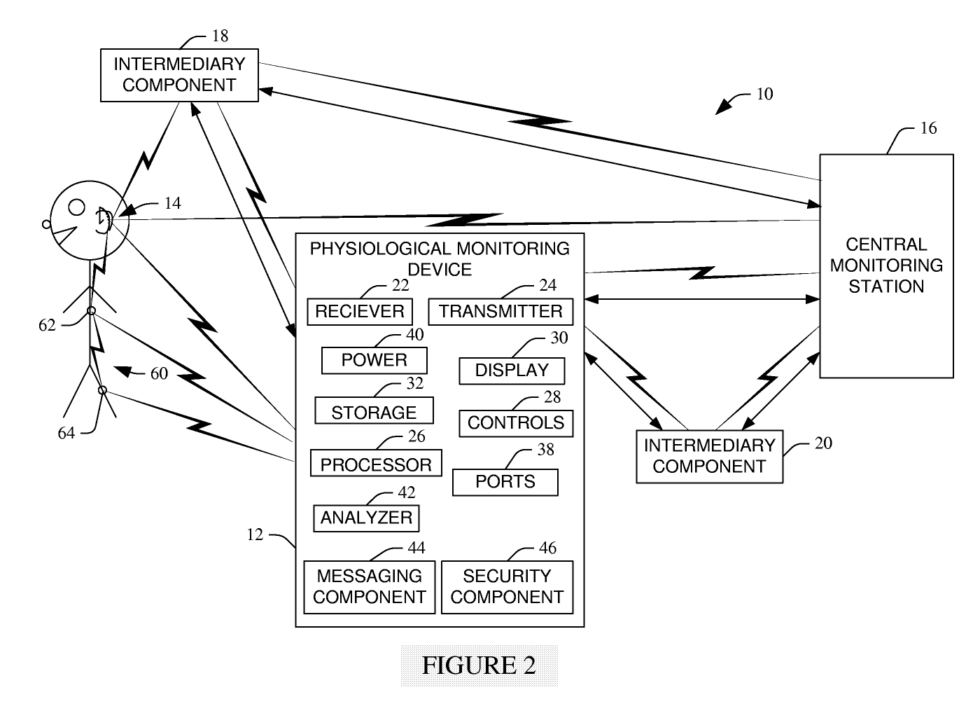

FIG. 2 illustrates another exemplary physiological monitoring device that communicates with an in-the-ear physiological measurement probe and other physiological monitoring equipment.

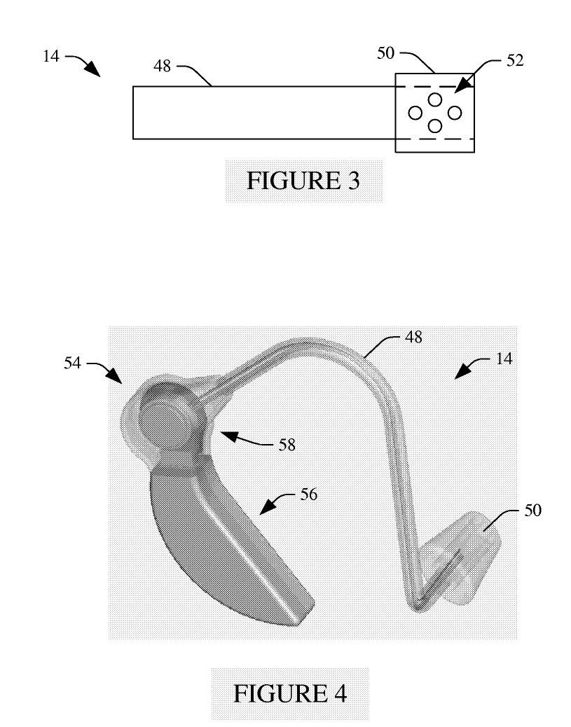

FIG. 3 illustrates an exemplary in-the-ear physiological measurement probe.

FIG. 4 illustrates an in-the-ear physiological measurement probe connected to a behind-the-ear supporting device.

FIG. 1 illustrates a physiological monitoring system (“system”) 10. The system 10 includes a physiological monitoring device 12, which is a mobile device that communicates with physiological measuring equipment (e.g., an in-the-ear probes, etc.) and devices (e.g., a central monitoring station, etc.) used in connection therewith. The physiological monitoring device 12 can be hand held or held by an ambulatory carrier. As described in detail below, the physiological monitoring device 12 can be used to intercept, display, validate and forward (via wire or wirelessly) physiological measurements continuously over a wireless network, or spot-check received physiological measurements obtained by an in-the-ear probe and communicate or download such measurements to a central monitoring station, send and receive information (e.g., physiological measurements, patient history, medical history, messages, notifications, alarms, etc.) to an authorized individual, the central monitoring station, another physiological monitoring device 12, etc., as well as various other activities.

As briefly discussed above, the physiological monitoring device 12 is used in connection with other physiological monitoring equipment. For example, an in-the-ear probe 14 (e.g., described in detail in connection with FIGS. 3-4 below) may be used at a hospital, a home, a nursing home, etc. to measure, record, and/or convey physiological parameters (e.g., non-invasive blood pressure, pulse, blood oxygen, temperature, perfusion, respiration, etc.) obtained by the probe 14 from within an ear of an individual. In such environments, the physiological parameters may be wirelessly transmitted (e.g., continuously, periodically at a predetermined rate, on-demand, upon occurrence of an event, etc.) from the probe 14 to a central monitoring station 16, an intermediate device 18 (e.g., a bedside monitor, a signal router, this physiological monitoring device 12 acting as a continuous bedside monitor, an input for a wired network that carries the measured parameters to the central station 16, etc.), etc. The physiological monitoring device 12 communicates (uni or bi-directionally) with the probe 14, the central monitoring system 16, optionally the intermediate device 18, and/or other devices such as a second intermediate component 20. Such communication can be through wired (e.g., Ethernet, USB, serial, parallel, FireWire, optical wave guides, telephone wire, coaxial cable, etc.) and/or wireless (e.g., radio frequency, infrared, optical, mechanical wave, magnetism, etc.) technologies.

Communication between the physiological monitoring device 12 and the probe 14 includes, but is not limited to, reception and/or retrieval via a receiver 22 of physiological measurements obtained by the probe 14, requests transmitted by a transmitter 24 to the probe 14 instructing the probe 14 to perform and/or send a physiological measurements) to the receiver 22, security indicia, device information such as a probe or device serial number, user identification, software/firmware upgrades for the probe 14, diagnostic applications to troubleshoot the probe 14, etc. In one instance, the foregoing communication is directly between the physiological monitoring device 12 and the probe 14, while in another instance, such communication between the physiological monitoring device 12 and the probe 14 is facilitated by the intermediary component 18 and/or other components.

The receiver 22 and/or the transmitter 24 can communicate over various communication mediums. For instance, the probe 14 may reside within a body area network 60. In this instance, the physiological monitoring device 12 can communicate within such network to interact with the probe 14, one or more physiological sensors 62 positioned on the patient, one or more emitters 64 positioned on the patient, local measurement devices measuring physiological parameters, the intermediary component 18, another physiological monitoring device 12, etc. The central monitoring station 16 may communicate over a network local to the facility, regional within the facility, and/or global to the community. The network may be part of or communicate with one or more larger networks such as a large area network (LAN), a wide area network (WAN), including the Internet, as well as other public and/or private networks. The central monitoring station 16 may communicate this selected information to the physiological monitoring device 12.

A processor 26 controls the receiver 22 and the transmitter 24. For instance, upon entering a communication range of the probe 14, the processor 26 can automatically invoke the receiver 22 to detect and capture information emitted by the probe 14, automatically invoke the transmitter 24 to send a request to the probe 14 for information stored therein, automatically invoke the transmitter 24 to perform measurements, establish a secure communication link with the probe 14, etc. Such requests may indicate which of a plurality of possible physiological parameters (e.g., blood pressure, blood oxygen, heart rate, respiration rate, temperature, etc.) to measure. The processor 26 can also automatically invoke conveyance of such information via the transmitter 24 to the central monitoring station 16 or the intermediary component 20.

Controls 28 provide various knobs, buttons, switches, sliders, audio receivers, tactile transducers, etc. to receive/send control commands from a user. For example, the controls 28 may include a mechanism with which the user can employ to invoke reception of information from the probe 14 and/or the intermediary component 18 by the receiver 22 or transmission of stored or received information by the transmitter 24 to the central monitoring station 16 and/or the intermediary component 20.

A display 30 visually presents received physiological measurements, or information from the central monitoring station, for observance by a user of the physiological monitoring device 12. In order to facilitate displaying such data, the display 30 can include, but is not limited to, one or more light emitting diodes, seven segment displays, a liquid crystal display, a flat panel display, a graphical user interface, etc. The controls 28 provide a user with a means for selecting information to present by the display 30 and configuring how the information is presented by the display 30.

Information, applications, etc. can be stored within the physiological monitoring device 12 in a storage component 32, which may include resident storage 34 and portable storage 36. Both the resident and the portable storages 34 and 36 can include various types of memory including volatile (e.g., various flavors of random access memory (RAM)) and non-volatile (e.g., various flavors of read only memory (ROM), flash memory, magnetic RAM (MRAM), non-volatile RAM (NVRAM), etc.) memory. The portable storage 36 can be used to transfer information stored therein from the physiological monitoring device 12 to the intermediary component 20 and/or the central monitoring station 16 and vice versa. For instance, flash memory (e.g., a universal serial bus (USB) based memory stick) can be inserted into a suitable port on the physiological monitoring device 12. Information can then be directly stored thereto or transferred/copied from the resident storage 34 to the portable storage 36. This can be achieved automatically upon inserting the portable storage 36 into a corresponding port, after manually selecting information to store within the portable storage 34, etc. The portable storage 36 can then be removed and inserted into a suitable port of the intermediary component 20 and/or the central monitoring station 16. The information can be automatically or manually retrieved from the portable storage 36. In another instance, the portable storage 36 can inserted into a suitable port of the intermediary component 20, the central monitoring station 16, etc. and applications, software/firmware, and/or other information can be loaded to the portable storage 36. The portable storage 36 can then be removed therefrom and inserted into a suitable port of the physiological monitoring device 12, wherein the information stored within the portable storage 36 can be moved to the resident storage 34 of the physiological monitoring device 12.

The physiological monitoring device 12 may also include one or more ports 38 for communicating information. The transmitter 24 can transmit information through the ports 38 to the central monitoring station, the intermediary component 20, etc. Suitable wired ports include, but are not limited to, Ethernet, USB, serial, parallel, FireWire, optical, and the like.

A power component 40 provides power to power the various components of the physiological monitoring device 12. The power component 40 can include one or more of a rechargeable and/or a non-rechargeable battery, a solar cell, a port for receiving DC from an AC to DC converter, an AC to DC converter, and/or the like.

In one instance, the ear probe 14 continuously transmits/emits information to the central monitoring station 16. When a user enters an area (e.g., a room) with the physiological monitoring device 12, the physiological monitoring device 12 receives real-time signals emitted by the probe 14 and presents a corresponding display via the display component 30. The user can view the information, validate the monitored vital signs, infer whether the monitored signals are accurate (e.g., by assessing signal quality, by comparing the information with previously stored information, ranges for typical information, etc.), etc. If a reading appears suspicious, the user can wait for signal quality to improve, take action to improve signal quality, or check the measurement with another instrument. When all readings appear to be correct, the user can provide the information and/or a validation indication to the central monitoring station 16.

In another instance, the physiological monitoring device 12 is used for on-demand monitoring or spot-checks. In this embodiment, the probe 14 is configured such that it does not continuously broadcast information. Rather, each time the user wants to view vital signs, the physiological monitoring device 12 requests and receives the current or stored vital signs using a low power short-range communication, such as Bluetooth, body coupled communications, and the like. Once the user has validated the readings, the physiological monitoring device 12 conveys the readings to the central monitoring station 16 with a higher power transmission with longer range. This conveyance can be achieved in real time by a radio frequency signal or the like, or the physiological monitoring device 12 can store the readings of one or more individuals in the storage component 32 and subsequently transfer the readings via a wireless or wired means to the central monitoring station 16.

In another instance, the physiological monitoring device 12 performs the above-discussed functions and further assumes additional functions that were previously performed by other devices. For example, the physiological monitoring device 12 may be able to communicate with staff members. In addition to communicating with other physiological monitoring devices 12 being used by other staff members, the physiological monitoring device 12 may be able to interact with personal data assistant, cell phones, beepers, telephones, email, etc. directly or through the central station 16. Through such devices, the physiological monitoring device 12 may be able to receive and deliver messages, notifications, medication schedules, documented delivery of medication, chart highlights, vitals validation, information, alarms, paging, etc. to a care-giver, a guardian, etc.

The physiological monitoring device 12 can also be used to memorialize, document, chart, etc. activity. Such activity can include, but is not limited to, physiological measurements and data derived thereform, the delivery of medications or medical assistance, the individual(s) administering the medications or medical assistance, the time such medications and assistance was given, scheduled procedures, medical history, unique identification, patient name, health insurance provider, family history, treating physicians, test results, etc.

FIG. 2 illustrates the physiological monitoring device 12 further having an analyzer 42, a messaging component 44, and a security component 46. The analyzer 42 analyzes information received from the probe 14 and generates trends, predicate future health, suggest treatments, etc. In addition, the analyzer 42 provides processing capabilities to process the received physiological measurements information. Suitable processing includes combining, averaging, weighting, etc. data. The raw and/or processed data can be presented to the user via alpha-numeric symbols, graphs, plots, audio, icons, trends, projections, historical comparisons, etc. on the display 30 and/or the central processing station 16.

The analysis can also be used to validate that received physiological measurements are within pre-stored ranges. For example, the analyzer 42 can assess signal quality and compare received measurements with acceptable ranges stored in the storage 32. Physiological measurements having insufficient signal quality, or that fall outside of expected physiological ranges may invoke the physiological monitoring device 12 to request re-transmissions of the information, request performance of new measurements, and/or sound an alarm. Such alarm may be a visual and/or audio alarm within the physiological monitoring device 12, an alarm at the central monitoring system, and/or other alarms. Such alarms may also include transmission of alarms, messages, notifications, etc. by the messaging component 44 to various individuals through various devices. Examples of suitable devices include, but are not limited to, another physiological monitoring device 12, a personal data assistant, a cell phone, beepers, a telephone, email, a beeper, a pager, etc.

The messaging component 44 may also send general messages, notifications, etc. to such individuals and/or equipment. The general messages, notifications, etc. may indicate that it is time to read a physiological measurement, administer a medication, replace or recharge a battery, etc. and/or that a physiological measurement has been acquired, a medication has been administered, an identification of the medical professional performing the activity, etc. In one instance, the messaging component 44 can be used as a walkie-talkie to allow the user to audibly communicate with an individual at the central monitoring station, an individual using a similar device, a cell phone, etc.

The security component 46 can be used to determine whether the user of the physiological monitoring device 12 is an authorized user. For instance, the physiological monitoring device 12 may require the user to enter a password or other identifying indicia that can be checked against predetermined authorized information. Likewise, security component 46 can validate the probe 14 to ensure that the probe 14 is associated with the correct individual (e.g., via unique identification entered by user or read from an RFID tag), that the physiological monitoring device 12 is authorized to communicate with the probe 14 (e.g., by checking unique identification, serial number, etc.), set up an encoded communication link with the probe 16, etc. For unauthorized use or communication, the physiological monitoring device 12 can lock the controls 28, dim the display 30, invoke the messaging component 44 to sound an alarm, etc.

FIG. 3 illustrates an exemplary configuration of the probe 14. In this configuration, the probe 14 is an in-the-ear (ITE) physiological measurement apparatus for measuring one or more physiological signals (e.g., blood pressure, pulse, blood oxygen, perfusion, temperature, respiration . . . ) from within an ear canal. The probe 14 includes a structure 48 that inserts into the ear canal. The structure 48 is suitably dimensioned to enter the ear canal to a suitable depth and adapts to various shaped ear canals (e.g., different curvatures). That is, the structure 48 is small in diameter compared to the diameter of the ear canal. In one instance, the structure 48 projects into the ear canal such that an end portion is positioned proximate to a bony region of the ear or other relatively quiet zone of the ear canal.

The end portion of the structure 48 residing in the ear canal may be fabricated with a spongy expandable material, or include an annular inflatable balloon 50. The spongy material or inflatable balloon 50 surrounds the end portion of the structure 48 (as illustrated) or suitable portions thereof. The spongy material or inflatable balloon 50 ideally supports one or more sensors 52 that are operatively coupled to a surface of the spongy material or balloon 50 and that measure physiological signals. Suitable sensors include light emitting diodes (LEDs), an infrared (IR) source, light detectors, a pressure transducer, a microphone, a speaker, an accelerometer, and a thermistor, for example. The sensors 52 are strategically positioned on the spongy material or balloon 50. For example, a light detecting sensor typically is positioned to minimize or prevent absorption of light not indicative of the physiological process under measurement (e.g., light from outside the ear, light emitted from another sensor located on the spongy material or balloon 50 . . . ). Although depicted as circular, the one or more sensors 46 can be any shape. Alternatively, the sensors could be mounted within the end portion of the structure 48 and could be moved into contact with the tissue once inserted into the ear.

The inflatable balloon 50 is inflated to position, or the spongy material positions the one or more sensors 52 proximate to appropriate tissue within the ear canal with ideal force and pressure to ensure close coupling of sensors with tissue but without causing decreased perfusion or blanching of the tissue. By way of example, the structure 48 is inserted such that the end portion with the spongy material or balloon 50 residing in the ear canal is in a bony region of the ear. The balloon 50 is inflated to position, or the spongy material positions the sensors 52 proximate to inner ear tissue to sense signals indicative of physiological states, including blood pressure, temperature, pulse, respiration, and blood oxygen, for example.

For adult humans, this includes inflating the balloon, or allowing the spongy material 50 to conform to the widely varying ear canal diameters from about 6 mm to about 13 mm. For neonates and small pediatrics, where the ear canal diameter various from about 4 mm in diameter to about 7 mm in diameter, smaller and shorter ITE devices are used. Typically, sensors for measuring blood oxygen are positioned proximate to ear canal tissue that is perfused with arterial blood supplied by branches of the External as well as the Internal Carotid Arteries, thus serving as a well perfused physiological site even if the body is experiencing peripheral shutdown due to shock or other conditions. Such sensors include an energy emitting means (e.g., an LED, an IR source . . . ) and an energy detecting means that detects energy transmission through the vascular tissue. In another example, a temperature sensor (e.g., a thermistor) is also positioned proximate to vascular tissue. In yet another example, sensors for sensing audio signals (e.g., a microphone) indicative of pulse pressure sounds, and/or respirations are suitably positioned in relatively quite regions of the ear canal to mitigate sensing extraneous audio signals (noise).

The inflatable balloon 50 must be used to facilitate non-invasively measuring blood pressure. For a non-invasive blood pressure measurement, the inflatable balloon 50 is inflated until it occludes blood flow in a portion of the ear proximate a blood pressure sensor(s) (e.g., a pressure transducer) operatively connected to the inflatable balloon 50. The pressure in the inflatable balloon 50 is then suitably released to deflate the inflatable balloon 50. A systolic and a diastolic blood pressure are obtained during inflation and/or deflation using an auscultatory approach (e.g., via a microphone operatively connected to the balloon 50) and/or an oscillometric approach (e.g., via optical sensing components attached to the balloon).

A continuous non-invasive blood pressure is measured by obtaining an initial blood pressure measure as describe above and then re-inflating the balloon 50 to a mean pressure. A servo mechanism periodically adjusts balloon pressure to locate a maximum pulse waveform amplitude indicative of mean blood pressure. As long as the derived mean pressure is relatively close to the initial pressure and/or the pulse waveform amplitudes are relatively close, the derived continuous systolic, diastolic, and mean blood pressure are calculated with high accuracy.

The structure 48 includes one or more passageways (not shown) that extend through the structure 48. Such passageways house sensor data, power, and control wires, provide a hermetically sealed channel for inflating/deflating the balloon 50, and/or allow pressure inside the ear to equalize with the environment during balloon inflation/deflation. In one instance, the structure 48 includes a channel for both housing sensor wiring and inflating/deflating the balloon 50. The channel isolates the wires from the inner ear environment, mitigating contamination of both the ear and the sensor wiring and provides a pressurized air conduit to the balloon 50. In another instance, the structure 48 includes separate channels for sensor wiring and inflating/deflating the balloon 50; one or more first channels house sensor wiring and a second channel provides the pressurized air conduit for inflating/deflating the balloon 50. In yet another example, an optional channel provides an ear pressure stabilizing mechanism that allows ear pressure to equalize with the environment during balloon inflation and/or deflation. This channel mitigates pressure build-up in the ear during balloon inflation and/or deflation and potential pain therefrom. The passageways can be variously shaped (e.g., oval, rectangular, irregular . . . ) to be conducive to the ear canal.

FIG. 4 illustrates the ITE probe 14 mechanically and electrically coupled with an exemplary behind-the-ear (BTE) device 54. In one instance, the structure 48 and the BTE device 54 are formed as a single unit, while in another instance the structure 48 and the BTE device 54 are detachably connected (as illustrated). Such attachment can be through a fastening means including a threaded connector, a snap, a set screw, an adhesive, a rivet, etc. An arm 56 provides support behind the ear and a battery 58 powers both devices. An optional sheath (not shown) can be placed over the structure 48 and/or balloon 50 to protect the ear and the structure/balloon/sensor assembly from contamination. In one aspect, the sheath can be semi-permeable to allow air flow, but prevent fluid from moving from one side of the sheath to the other side. In another aspect, the sheath prevents substantially all matter from moving from one side of the sheath to the other side. The structure/balloon/sensor assembly can be disposable, washable, and/or sterilizeable.

In another embodiment, the in the ear structure 48 houses a smaller battery, a low powered transmitter, a processor and the like. A separate unit carried by the patient houses a receiver for the low power signals, a higher power transmitter which communicates with the physiological monitor device 12, the central station 16, etc., a larger battery, and, optionally, a processor, memory, and action appropriate components and software.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A portable physiological monitoring device, comprising:
a receiver that wirelessly receives physiological measurements from each of a plurality of in-the-ear probes upon entering a communication range of one of the in-the-ear probes; and
a display that presents one or more of the physiological measurements.

2. The physiological monitoring device as set forth in claim 1, further including a transmitter for conveying the received physiological measurements to at least one of a central monitoring station and an intermediary component.

3. The physiological monitoring device as set forth in claim 2, wherein the physiological measurements are conveyed by the transmitter through at least one of a wireless port, a wired port, and portable storage.

4. The physiological monitoring device as set forth in claim 2, further including a messaging component that sends at least one of an alarm, a message, and a notification to at least one of the central monitoring station, another physiological monitoring device, an intermediary component, a personal data assistant, a cell phone, a beeper, a telephone, an email address, and a pager.

5. The physiological monitoring device as set forth in claim 1, wherein the receiver automatically captures physiological measurements continuously emitted by the in-the-ear probe.

6. The physiological monitoring device as set forth in claim 1, wherein the receiver captures physiological measurements measured and emitted by the in-the-ear probe on-demand.

7. The physiological monitoring device set forth in claim 1, further including a storage component for storing physiological measurements.

8. The physiological monitoring device as set forth in claim 7, wherein the storage component includes portable storage for storing and transferring physiological measurements from the device to at least one of a central monitoring station and an intermediary component.

9. The physiological monitoring device as set forth in claim 1, further including a display that presents processed data through alphanumeric symbols, graphs, plots, audio, icons, trends, projections, and historical comparisons.

10. The physiological monitoring device as set forth in claim 1, further including an analyzer that analyzes the received physiological measurements and that performs at least one of: aggregates data, summarizes data, generates trends, predicts future health, and suggest treatments.

11. The physiological monitoring device as set forth in claim 1, further including a control through which a user inputs commands to validate received physiological measurements and to perform other control functions.

12. The physiological monitoring device as set forth in claim 1, further including a security component that performs at least one of: authorizes use of the physiological monitoring device, and creates secure communication links between the monitoring device and each of probes from which physiological measurements are to be reviewed.

13. The physiological monitoring device as set forth in claim 1, wherein the physiological measurements include one or more of blood pressure, respiration, perfusion, blood oxygen, pulse rate, activity, and body temperature.

14. The physiological monitoring device as set forth in claim 1, wherein the in-the-ear probe resides within a body area network and the receiver further receives information transmitted from at least one of a sensor and an emitter communicating within the body area network.

15. The physiological monitoring device as set forth in claim 1, further including a transmitter that conveys one or more of software/firmware upgrades to the-ear probe and executing diagnostics that troubleshoot the-ear probe.

16. A method for conveying physiological parameters measured by each of a plurality of in-the-ear probes to a monitoring station through a portable physiological monitoring device, comprising:
receiving physiological parameters measured by an in-the-ear probe with the portable physiological monitoring device;
storing, processing and displaying the physiological measurements with the portable physiological monitoring device;
transferring the physiological measurements to a monitoring station either directly or through an intermediary component.

17. The method as set forth in claim 16, further including with the portable physiological monitoring device invoking the in-the-ear probe to at least one of perform a physiological measurement and emit a measured physiological parameter on-demand.

18. The method as set forth in claim 16, wherein the physiological measurements include one or more of blood pressure, respiration, perfusion, blood oxygen, pulse rate, activity, and body temperature.

19. The method as set forth in claim 16, further including with the portable physiological monitoring device sending at least one of an alarm, a message, and a notification regarding the physiological measurements to at least one of a central monitoring station, another physiological monitoring device, an intermediary component, a personal data assistant, a cell phone, a beeper, a telephone, an email address, and a pager.

20. The method as set forth in claim 16, further including storing physiological measurements for a plurality of the probes associated with different individuals and subsequently conveying one or more of the stored physiological measurements to a central monitoring station.

21. The method as set forth in claim 16, wherein the operator uses the portable physiological monitoring device to acquire measured physiological parameters from each of a plurality of patients, and wherein each of the plurality of patients is associated with at least one of the plurality of in-the-ear probes, and further including:
entering a communication range of one of the plurality of in-the-ear probes corresponding to one of the plurality of patients;
receiving physiological parameters measured by the one of the in-the-ear probes with the portable physiological monitoring device;
storing the physiological measurements of the one of the plurality of patients within the portable physiological monitoring device; and
repeating the steps of entering a communication range for each of the plurality of probes, receiving the physiological parameters measured by each of the plurality of probes for each of the plurality of patients, and storing the acquired physiological measurements associated with each of the plurality of patients within the portable physiological monitoring device.

22. The method as set forth in claim 21, further including:
utilizing control through which received physiological measurements are validated.

23. The method as set forth in claim 21, further including:
conveying the physiological measurements for two or more of the plurality of patients to a central monitoring station after upon storing the physiological measurements for the plurality of patients in the portable physiological monitoring device.

24. An apparatus for performing the method according to claim 16.

25. A portable physiological parameter monitoring device that stores and displays measured physiological parameters for each of a plurality of patients and a plurality of probes each coupled with one of the patients to measure physiological parameters of the coupled patient, each probe including a wireless communication transmitter and one or more physical parameter measuring devices, which can be carried by an operator from patient to patient, the portable physiological monitoring device, comprising:
a receiver for receiving measured physiological parameters from a proximate one of the probes with which wireless communications have been temporally established; and
a display on which a human readable representation of the measured physiological parameters is displayed.

26. The portable physiological parameter monitoring device as set forth in claim 25, further including:
controls through which the operator enters information and commands; and
a transmitter through which at least entered information and commands are communicated to at least one of the central station, an intermediary component, and another portable physiological parameter monitoring device.
